(19) United States
(12) Patent Application Publication
(10) Pub. No.: US 2013/0261426 A1
(43) Pub. Date: Oct. 3, 2013

(54) PHOTOACOUSTIC INSPECTION PROBE AND PHOTOACOUSTIC INSPECTION APPARATUS

(71) Applicant: FUJIFILM Corporation, Tokyo (JP)

(72) Inventors: Kaku IRISAWA, Kanagawa-ken (JP); Kazuhiro TSUJITA, Kanagawa-ken (JP)

(21) Appl. No.: 13/909,525

(22) Filed: Jun. 4, 2013

Related U.S. Application Data
(63) Continuation of application No. PCT/JP2011/006931, filed on Dec. 12, 2011.

(30) Foreign Application Priority Data
Dec. 10, 2010 (JP) 2010-275389
Nov. 28, 2011 (JP) 2011-258578

(51) Int. Cl. A61B 5/00

(52) U.S. Cl. CPC A61B 5/0095
USPC 600/407

(57) ABSTRACT

A probe employed in photoacoustic inspection is equipped with: a light emitting section that emits a light beam onto a subject; and an electroacoustic converting section that converts photoacoustic waves into electrical signals. The light emitting section is configured to emit light onto an emission range on the subject that includes the entirety of a corresponding region of the subject that corresponds to the electroacoustic converting section, the minimum distance between the outer peripheral edge of the corresponding region and the outer peripheral edge of the emission range being 5 mm or greater. The present invention enables detection of photoacoustic waves with a higher S/N ratio, in photoacoustic inspection that utilizes the photoacoustic effect.

### TECHNICAL FIELD

The present invention is related to a probe employed in photoacoustic inspections that detects photoacoustic waves which are generated within the body of a subject by light being irradiated on the subject and generates photoacoustic images. The present invention is also related to a photoacoustic inspection apparatus equipped with the probe.

### BACKGROUND ART

Ultrasound imaging, in which ultrasound images are generated by detecting ultrasonic waves which are reflected within the body of a subject by the ultrasonic waves being emitted within the body of the subject, to enable display of morphological tomographic images, is known as a conventional method for obtaining morphological tomographic images of the interior of the body of a subject. Meanwhile, there have been recent advancements in the development of apparatuses which are capable of displaying not only morphological tomographic images but also functional tomographic images in examinations of subjects. One such apparatus is that which utilizes the photoacoustic analysis method. The photoacoustic analysis method emits light having a predetermined wavelength (visible light, near infrared light, or intermediate infrared light, for example) onto a subject, detects photoacoustic waves, which are elastic waves generated as a result of a specific substance within the subject absorbing the energy of the light, and quantitatively measures the concentration of the specific substance. The specific substance within the subject is glucose, hemoglobin, etc., which is included in blood. The technique that detects photoacoustic waves and generates photoacoustic images based on detected signals is referred to as PAI (Photo Acoustic Imaging) or PAT (Photo Acoustic Tomography).

The following problem exists in conventional photoacoustic imaging that utilizes the photoacoustic effect. The intensity of light emitted onto a subject is significantly attenuated by absorption and scattering while propagating through the interior of the subject. In addition, the intensity of photoacoustic waves which are generated within the subject based on the emitted light is also attenuated by absorption and scattering while propagating through the interior of the subject. Accordingly, it is difficult to obtain information regarding deep portions of the subject by photoacoustic imaging. A possible solution to this problem is to increase the amount of light emitted onto the interior of the subject, to cause the generated photoacoustic waves to become larger.

However, in the case that the subject is living tissue, an MPE (Maximum Permissible Exposure) per unit area is defined in order to prevent living tissue systems from being damaged by the energy of emitted light. For this reason, the MPE will be the upper limit, even if the amount of emitted light is increased.

Therefore, Japanese Unexamined Patent Publication No. 2010-125260 discloses an apparatus that emits light such that the intensity distribution thereof will be uniform, in order to enable detection of photoacoustic waves with a high S/N ratio, even if the amount of emitted light is the MPE or less.

### DISCLOSURE OF THE INVENTION

However, in the method adopted by the apparatus of Japanese Unexamined Patent Publication No. 2010-125260 only causes the intensity distribution of light to become uniform, and does not consider the emission range of the light. Therefore, there is a problem that cases may occur in which photoacoustic waves cannot be detected with a sufficiently high S/N ratio.

The present invention has been developed in view of the foregoing circumstances. It is an object of the present invention to provide a photoacoustic inspection probe and a photoacoustic inspection apparatus that enable detection of photoacoustic waves with a higher S/N ratio, in photoacoustic inspection that utilizes the photoacoustic effect.

A photoacoustic inspection probe of the present invention that solves the aforementioned problem is a photoacoustic inspection probe for use in photoacoustic inspection that emits a light beam onto a subject, guides the light beam to the subject, detects photoacoustic waves which are generated within the subject by the light beam being irradiated thereon, converts the photoacoustic waves to electrical signals, and performs inspection based on the electrical signals, comprising:

a light emitting section that emits the light beam onto the subject; and

an electroacoustic converting section that converts the photoacoustic waves into electrical signals;

the light emitting section being configured to emit light onto an emission range on the subject that includes the entirety of a corresponding region of the subject that corresponds to the electroacoustic converting section, the minimum distance between the outer peripheral edge of the corresponding region and the outer peripheral edge of the emission range being 5 mm or greater.

In the present specification, the expression “corresponding region” refers to a contact region in the case that the electroacoustic converting section and the subject directly contact each other, and refers to a region on the subject corresponding to a detecting surface of the electroacoustic converting section through an acoustic rectifying layer in the case that the acoustic rectifying layer is present between the electroacoustic converting section and the subject.

The expression “outer peripheral edge of the emission range” refers to a collection of positions at which the intensity of the light beam on the subject becomes half the average intensity of the light beam at the outer peripheral edge of the corresponding region.

The expression “minimum distance between the outer edge of the corresponding region and the outer peripheral edge of the emission range” refers to the minimum width of an annular region between the outer peripheral edge of the emission range and the outer peripheral edge of the corresponding region.

In the photoacoustic inspection probe of the present invention, it is preferable for:

the maximum distance between the outer peripheral edge of the corresponding region and the outer peripheral edge of the emission range to be 20 mm or less.

In the present specification, the expression “maximum distance between the outer peripheral edge of the corresponding region and the outer peripheral edge of the emission” refers to the maximum width of an annular region between the outer peripheral edge of the emission range and the outer peripheral edge of the corresponding region.

It is preferable for the light emitting section to be equipped with a plurality of light guiding sections that branch the light beam, arranged at constant intervals. Alternatively, it is preferable for the light emitting section to be equipped with a plurality of combinations of a light guiding section that branches the light beam and a circular lens. As a further alternative, it is preferable for the light emitting section to be equipped with a combination of a plurality light guiding sections provided at constant intervals that branch the light beam, and a rectangular lens.

It is preferable for the light emitting section to be equipped with a light diffusing section for causing light having a substantially uniform intensity to be emitted within the emission range.

It is preferable for the emission axis of the light beam emitted by the light emitting section to be inclined at an angle within a range from 5 through 45 degrees toward the corresponding region with respect to a direction perpendicular to the corresponding region.

It is preferable for the light emitting section to be a pair of light guiding plates which are provided to face each other with the electroacoustic converting section interposed therebetween. In this case, it is preferable for the light guiding plates to have light diffusing sections at the end portions thereof through which light is output. In addition, it is preferable for the end portions of the light guiding plates through which the light beam is output to be cut so as to have a slope.

Further, a photoacoustic inspection apparatus of the present invention comprises:

a light source that generates a light beam to be emitted onto a subject;

a light emitting section that emits the light beam onto the subject;

an electroacoustic converting section that converts photoacoustic waves generated within the body of the subject by the light beam being emitted thereon and converts the photoacoustic waves into electrical signals; and

an image generating section that generates photoacoustic images based on the electrical signals;

the light emitting section being configured to emit light onto an emission range on the subject that includes the entirety of a corresponding region of the subject that corresponds to the electroacoustic converting section, the minimum distance between the outer peripheral edge of the corresponding region and the outer peripheral edge of the emission range being 5 mm or greater.

In the photoacoustic inspection apparatus of the present invention, it is preferable for:

the maximum distance between the outer peripheral edge of the corresponding region and the outer peripheral edge of the emission range to be 20 mm or less.

In addition, it is preferable for the light emitting section to be light guiding plates which are arranged to face each other with the electroacoustic converting section interposed therebetween.

The photoacoustic inspection probe and the photoacoustic inspection apparatus of the present invention are to be employed in photoacoustic inspection that utilizes the photoacoustic effect, and are equipped with the light emitting section that emits the light beam onto the subject and the electroacoustic converting section that converts the photoacoustic waves into electrical signals. Particularly, the light emitting section is configured to emit light onto an emission range on the subject that includes the entirety of a corresponding region of the subject that corresponds to the electroacoustic converting section, the minimum distance between the outer peripheral edge of the corresponding region and the outer peripheral edge of the emission range being 5 mm or greater. Therefore, photoacoustic waves having greater intensities can be generated from tissue systems in cases that the light energy density is the same. As a result, photoacoustic waves can be detected with higher S/N ratios in photoacoustic inspection that utilizes the photoacoustic effect.

### BRIEF DESCRIPTION OF THE DRAWINGS

FIG. 1 is a schematic diagram that illustrates the configuration of a photoacoustic imaging apparatus according to an embodiment of the present invention.

FIG. 2 is a schematic diagram that illustrates the configuration of an image generating section of FIG. 1.

FIG. 3 is a schematic diagram that illustrates an ultrasound probe.

FIG. 4 is a schematic sectional diagram that illustrates the ultrasound probe and living tissue when generating a photoacoustic image.

FIG. 5A is a schematic diagram for explaining the positional relationship between the outer peripheral edge of a corresponding region related to one dimensionally arranged converting elements and the outer peripheral edge of an emission range that includes the corresponding region.

FIG. 5B is a schematic diagram for explaining the positional relationship between the outer peripheral edge of a corresponding region related to one dimensionally arranged converting elements and the outer peripheral edge of an emission range that includes the corresponding region.

FIG. 5C is a schematic diagram for explaining the positional relationship between the outer peripheral edge of a corresponding region related to one dimensionally arranged converting elements and the outer peripheral edge of an emission range that includes the corresponding region.

FIG. 5D is a schematic diagram that illustrates an example of a light emitting section.

FIG. 6 is a schematic diagram that illustrates the positional relationship between the outer peripheral edge of a corresponding region related to two dimensionally arranged converting elements and the outer peripheral edge of an emission range that includes the corresponding region.

FIG. 7A is a schematic diagram that illustrates an example of a light emitting section configured to be capable of emitting light from above an electroacoustic converting section.

FIG. 7B is a schematic diagram that illustrates another example of a light emitting section configured to be capable of emitting light from above an electroacoustic converting section.

FIG. 7C is a schematic diagram that illustrates still another example of a light emitting section configured to be capable of emitting light from above an electroacoustic converting section.

FIG. 7D is a schematic sectional diagram that illustrates an example of a light emitting section that utilizes a light guiding plate.

FIG. 7E is a schematic sectional diagram of the ultrasound probe of FIG. 7D viewed from an elevation direction.

FIG. 7F is a schematic sectional diagram that illustrates another example of a light emitting section that utilizes a light guiding plate.

FIG. 7G is a schematic sectional diagram that illustrates still another example of a light emitting section that utilizes a light guiding plate.

FIG. 8A is a schematic diagram that illustrates the configuration of an apparatus according to an embodiment of the present invention.

FIG. 8B is a schematic diagram that illustrates the configuration of an apparatus according to an embodiment of the present invention.

FIG. 9 is a graph that illustrates measurement results obtained by an embodiment of the present invention.

### BEST MODE FOR CARRYING OUT THE INVENTION

Hereinafter, embodiments of the present invention will be described with reference to the attached drawings. However, the present invention is not limited to the embodiments to be described below. Note that the dimensional ratios of the constituent elements in the drawings are different from the actual dimensional ratios in order to facilitate visual understanding.

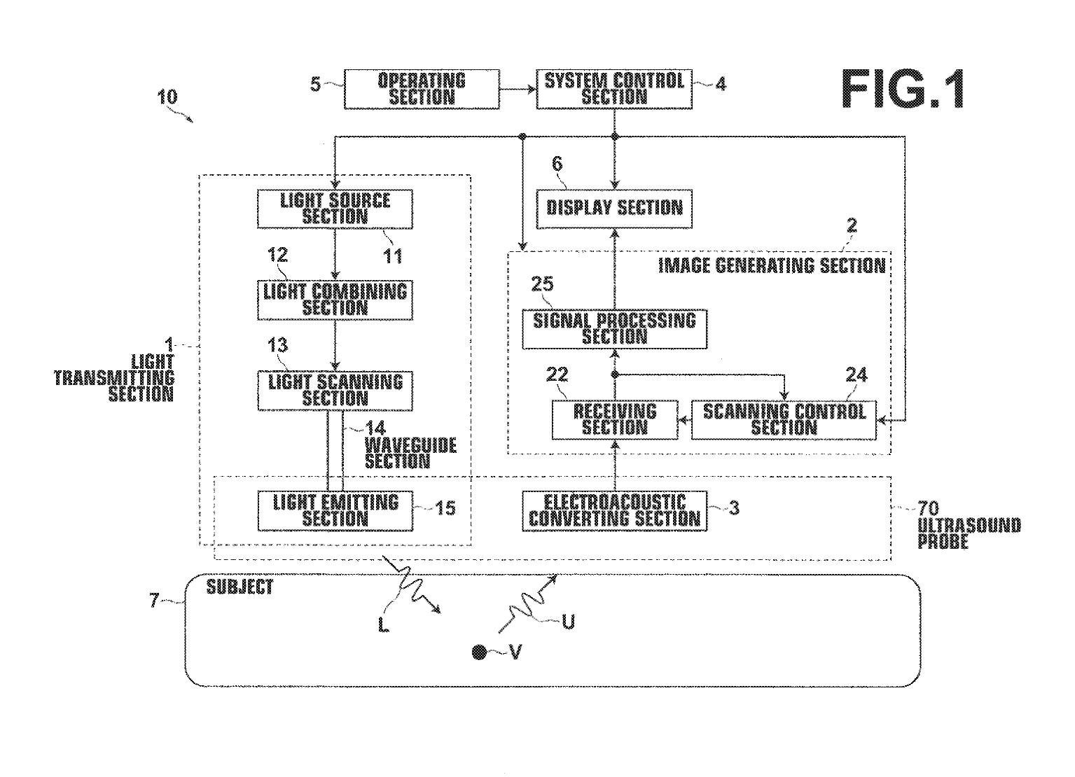

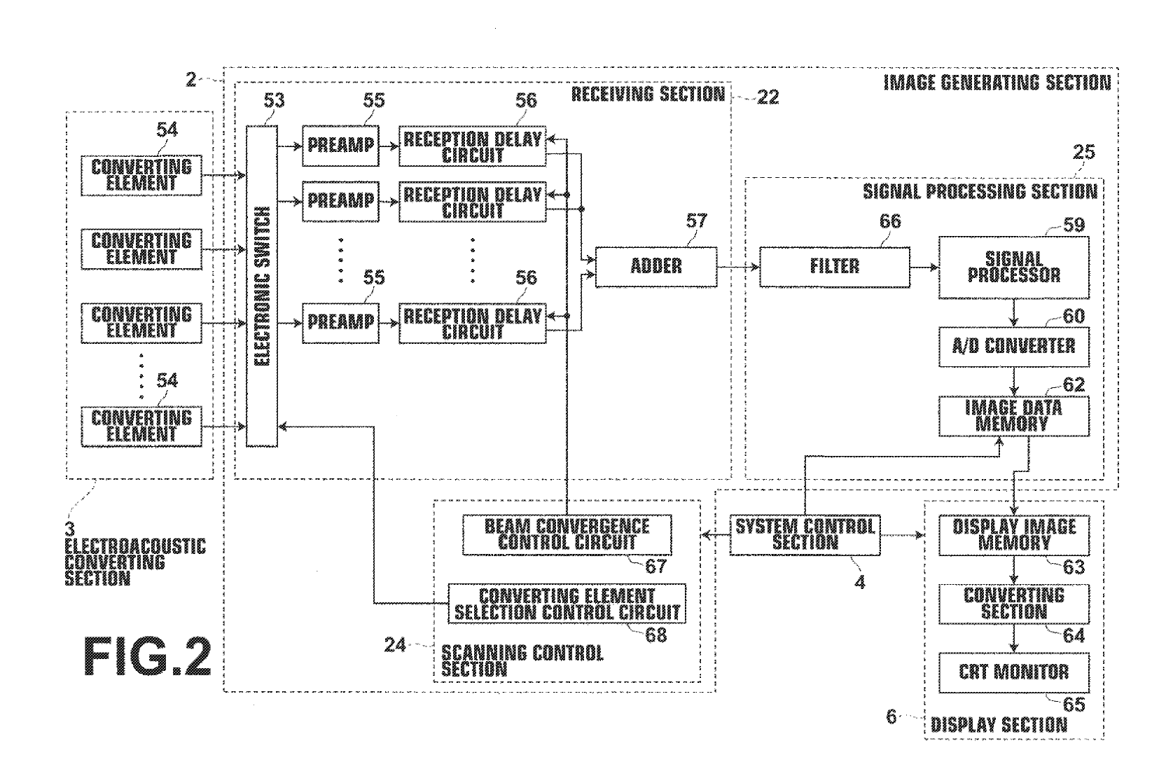

An embodiment of a photoacoustic inspection apparatus 10 equipped with a photoacoustic inspection probe of the present invention will be described. FIG. 1 is a block diagram that illustrates the schematic structure of the entirety of the Photoacoustic inspection apparatus 10 of the present embodiment. FIG. 2 is a block diagram that illustrates the configuration of an image generating section 2 of FIG. 1.

The photoacoustic inspection apparatus 10 of the present embodiment is equipped with: a light transmitting section 1 that generates a measuring light beam L that includes a specific wavelength component and emits the measuring light beam L onto a subject 7; an image generating section 2 that detects photoacoustic waves U, which are generated within the body of the subject by the measuring light beam L being emitted onto the subject 7, and generates photoacoustic image data of a desired cross section; an electroacoustic converting section 3 that converts acoustic signals to electric signals; a display section 6 that displays the photoacoustic image data; an operating section 5 through which an operator inputs patient data and imaging conditions for the apparatus; and a system control section 4 that controls each component and the apparatus as a whole.

The light transmitting section 1 is equipped with: a light source section 11 having a plurality of light sources that output light beams having different wavelengths; a light combining section 12 that combines the light beams having a plurality of wavelengths into a single optical axis; a multiple channel waveguide section 14 that guides the light beams to the body surface of the subject 7; a light scanning section 13 that switches the channel of the waveguide section 14 to be utilized and performs scanning; and a light emitting section 15 that outputs the light beams provided by the waveguide section 14 toward the subject 7.

The light source section 11 has one or more light sources that emit light beams having predetermined wavelengths. Light emitting elements such as semiconductor lasers (LD's), light emitting diodes (LED's), solid state lasers, gas lasers, etc. that emit specific wavelength components or single color light beams that include the specific wavelength components may be employed as the light sources. It is preferable for a light source 16 to output pulsed light having a pulse width within a range from 1 nsec to 100 nsec as the measuring light beam. The wavelength of the measuring light beam is determined as appropriate according to the light absorbing properties of the substance within the subject's body which is the target of measurement. The optical absorption properties of hemoglobin differ according to the state thereof (oxidized hemoglobin, reduced hemoglobin, methemoglobin, carbon gas hemoglobin, etc.), but hemoglobin generally absorbs light having wavelengths from 600 nm to 1000 nm. Accordingly, in the case that the measurement target is hemoglobin within an organism (that is, a case in which blood vessels are imaged), it is generally preferable for the wavelength of the measuring light beam to be within a range from 600 nm to approximately 1000 nm. It is further preferable for the wavelength of the measuring light beam to be within a range from700 nm to 1000 nm, from the viewpoint of being able to reach deep portions of a subject M. The output of the measuring light beam is preferably within a range from 10 μJ/cm² to several tens of μJ/cm² from the viewpoint of transmission loss of light and photoacoustic waves, photoacoustic conversion efficiency, detection sensitivity of presently available detectors, etc. It is further preferable for repetition of the pulsed light output to be 10 Hz or greater from the viewpoint of image construction speed. In addition, the measuring light beam may be a pulse row, in which a plurality of the pulsed light beams are arranged.

As a more specific example, a Nd:YAG laser (light emission wavelength: 1064 nm), which is a type of solid state laser, or a He—Ne gas laser (light emission wavelength: 633 nm), which is a type of gas laser, may be employed to form a laser light beam having a pulse width of approximately 10 nsec in the case that the concentration of hemoglobin in the subject 7 is to be measured. In the case that a miniature light emitting element such as a LD or a LED is employed, an element that employs materials such as InGaAlP (light emission wavelength: 550 nm to 650 nm), GaAlAs (light emission wavelength: 650 nm to 900 nm), and InGaAs or InGaAsP (light emission wavelength: 900 nm to 2300 nm) may be utilized. In addition, light emitting elements that employ InGaN and emit light having wavelengths of 550 nm or less are recently being put into practical use. Further, an OPO (Optical Parametrical Oscillator) laser that employs a non linear optical crystal to enable wavelengths to be varied may be employed.

The light combining section 12 causes the light beams having different wavelengths emitted by the light source section 11 to be overlapped along a single optical axis. Each light beam is converted to a collimated light beam by a collimating lens, and then the optical axis thereof is matched by a perpendicular prism or a dichroic prism. A comparatively small combining optical system can be constituted by such a configuration. Alternatively, commercially available multiplex wavelength combiner/separators may be employed. In the case that a light source capable of continuously changing wavelengths such as the aforementioned OPO laser is utilized in the light source section 11, the light combining section 12 is not necessary.

The waveguide section 14 guides the measuring light beam output by the light combining section 12 to the light emitting section 15. Optical fibers or thin film optical waveguides are employed in order to perform efficient light propagation. Alternatively, direct spatial propagation is also possible. Here, the waveguide section 14 is constituted by a plurality of optical fibers 71. A specific optical fiber 71 is selected from among the plurality of optical fibers 71, and light is irradiated onto the subject 7 by the selected optical fiber 71. Note that although not clearly illustrated in FIG. 1, the optical fibers may be utilized in combination with optical systems such as optical filters and lenses.

The light scanning section 13 scans the subject 7 with the measuring light beam, by sequentially selecting optical fibers from among the plurality of optical fibers 71 which are arranged in the waveguide section 14 and emitting the measuring light beam.

In the present embodiment, the light emitting section 15 is constituted by a plurality of output end portions of the plurality of optical fibers 71. The light emitting section 15 and the electroacoustic converting section 3 constitute an ultrasound probe 70. The plurality of output end portions of the plurality of optical fibers 71 are arranged along the periphery of the electroacoustic converting section 3. In the case that a plurality of converting elements 54 that constitute the electroacoustic converting section 3 are formed by a transparent material, the light emitting section 15 may be provided to irradiate the entirety of the converting elements from above the converting elements 54. Note that the plurality of output end portions of the plurality of optical fibers 71 form a flat surface, a convex surface, or a concave surface along with the plurality of converting elements 54 that constitute the electroacoustic converting section 3. Here, a case will be described in which the surface formed by the plurality of output portions and the converting elements 54 is a flat surface.

The electroacoustic converting section 3 is constituted by a plurality of very fine converting elements 54 which are arranged either one dimensionally or two dimensionally. The converting elements 54 are piezoelectric elements formed by a piezoelectric ceramic or a polymer film such as PVDF (polyvinylidene fluoride). The electroacoustic converting section 3 receives photoacoustic waves U which are generated within the subject 7 by the light emitting section 15 emitting the measuring light beam L thereon. The converting elements 54 function to convert the received photoacoustic waves U into electrical signals. The electroacoustic converting section 3 is configured to be small and lightweight, and is connected to a receiving section 22 to be described later by a multiple channel cable. The electroacoustic converting section 3 is selected from a sector scanning type, a linear scanning type, and a convex scanning type according to body parts to be diagnosed. The electroacoustic converting section 3 may be equipped with an acoustic rectifying layer in order to efficiently transmit the photoacoustic waves U. Generally, the acoustic impedances of piezoelectric materials and living tissue greatly differ. Therefore, in cases that a piezoelectric material directly contacts living tissue, reflection at the interface thereof becomes great, and photoacoustic waves cannot be efficiently transmitted. However, photoacoustic waves can be efficiently transmitted by inserting an acoustic rectifying layer formed by a substance having an intermediate acoustic impedance between the piezoelectric material and the living tissue. Examples of materials of the acoustic rectifying layer include epoxy resin and quartz glass.

The image generating section 2 of the photoacoustic inspection apparatus 10 is equipped with: a receiving section 22 that selectively drives the plurality of converting elements 54 that constitute the electroacoustic converting section 3, imparts predetermined delays to electrical signals output by the electroacoustic converting section 3, and generates received signals by performing phase matched addition; a scanning control section 24 that controls the selective driving of the converting elements 54 and the delay time imparted by the receiving section 22; and a signal processing section 25 that administers various processes on the received signals obtained from the receiving section 22.

As illustrated in FIG. 2, the receiving section 22 is equipped with an electronic switch 53, preamps 55, reception delay circuits 56, and an adder 57.

The electronic switch 53 continuously selects a predetermined number of adjacent converting elements 54 when receiving photoacoustic waves during photoacoustic scanning. For example, in the case that the electroacoustic converting section 3 is constituted by 192 converting elements CH1 through CH192 in an array, the arrayed converting elements are divided into three regions, which are: Area 0 (a region from converting element CH1 through converting element 64) ; Area 1 (a region from converting element CH65 through converting element 128); and Area 2 (a region from converting element CH129 through converting element 192). The arrayed converting elements constituted by N converting elements are handled as groups (areas) of n (n<N) adjacent converting elements in this manner. In the case that imaging operations are executed for each area, the need to connect preamps and A/D converting boards to converting elements for each channel is obviated, thereby simplifying the configuration of the ultrasound probe 70 and preventing cost increases. In addition, in the case that a plurality of optical fibers are provided such that light can be emitted individually onto each area, the light output for each emission can be kept low. Therefore, an advantage is obtained, that the need to employ a costly high output light source is obviated. The electrical signals obtained by each of the converting elements 54 are provided to the preamps 55.

The preamps 55 amplify the weak electrical signals received from the selected converting elements 54, to secure a sufficient S/N ratio.

The reception delay circuit 56 imparts a delay time to the electrical signals corresponding to the photoacoustic waves U obtained from the converting elements 54 selected by the electronic switch 53, in order to form a convergent received beam in which the phases of the photoacoustic waves U from a predetermined direction are matched.

The adder 57 adds the electrical signals output from the plurality of channels and delayed by the reception delay circuit 56 to organize the electrical signals into a single received signal. The addition adds acoustic signals from a predetermined depth by phase matched addition, and sets a reception convergence point.

The scanning control section 24 is equipped with: a beam convergence control circuit 67 and a converting element selection control circuit 68. The converting element selection control circuit 68 provides position data of the predetermined number of converting elements 54, to be selected by the electronic switch 53 during signal reception, to the electronic switch 53. Meanwhile, the beam convergence control circuit 67 provides delay time information for the predetermined number of converting elements 54 to form the reception convergence point to the reception delay circuit 56.

The signal processing section 25 is equipped with: a filter 66; a signal processor 59; an A/D converter 60; and an image data memory 62. The electrical signals output from the adder 57 of the receiving section 22, are processed by the filter 66 of the signal processing section 25 such that unnecessary noise is removed therefrom. Thereafter, the signal processor 59 administers logarithmic conversion on the amplitude of the received signals to relatively emphasize weak signals. Generally, signals received from the subject 7 have an amplitude with a wide dynamic range of 80 dB or greater. Amplitude compression to emphasize weak signals is necessary in order to display these signals on common CRT monitors, which have dynamic ranges of approximately 23 dB. Note that the filter 66 has bandwidth transmission properties, and has a mode that extracts basic waves within the received signals and a mode that extracts high frequency components. In addition, the signal processor 59 performs envelope detection on the logarithmically converted received signals. The A/D converter 60 administers A/D conversion to the signals output by the signal processor 59, to generate photoacoustic image data corresponding to single lines. The photoacoustic image data corresponding to the single lines are stored in the image data memory 62.

The image data memory 62 is a memory circuit that stores the photoacoustic image data generated in the manner described above. Under control of the system control section 4, data regarding cross sections are read out from the image data memory 62, and photoacoustic image data of the cross sections are generated by spatial interpolation during the readout.

The display section 6 is equipped with: a display image memory 63; a photoacoustic image data converter 64; and a CRT monitor 65. The display image memory 63 is a buffer memory that temporarily stores photoacoustic image data to be displayed on the CRT monitor 65. The photoacoustic image data corresponding to single lines from the image data memory 62 are combined into a single frame in the display image memory 63. The photoacoustic image data converter 64 performs D/A conversion and television format conversion on the combined image data read out from the display image memory 63. The output of the photoacoustic image data converter 64 is displayed by the CRT monitor 65.

The operating section 5 is equipped with a keyboard, a trackball, a mouse, etc. on an operating panel, and is employed by an operator to input necessary information, such as patient data, imaging conditions, and cross sections to be displayed.

The system control section 4 is equipped with a CPU (not shown) and a memory circuit (not shown). The system control section 4 controls each of the components, such as the light transmitting section 1, the image generating section 2, the display section 6, and the system as a whole, according to command signals input via the operating section 5. Particularly, command signals input by the user via the operating section 5 are stored in the internal CPU.

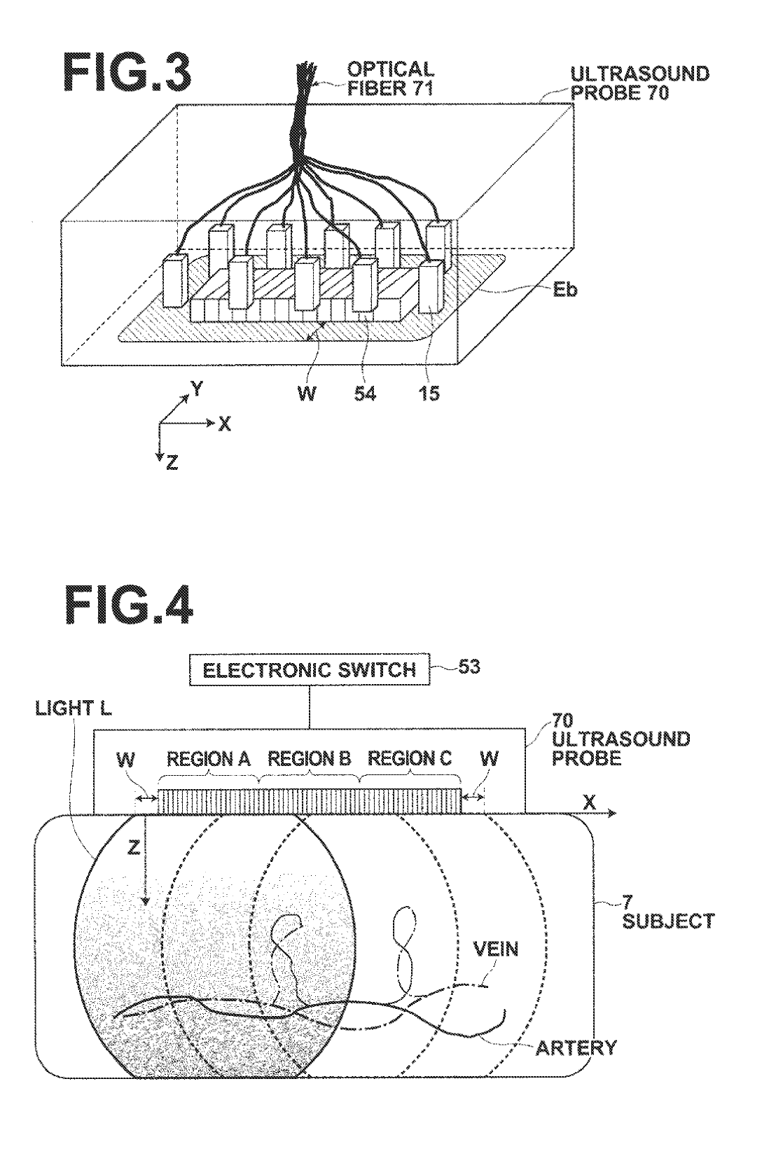

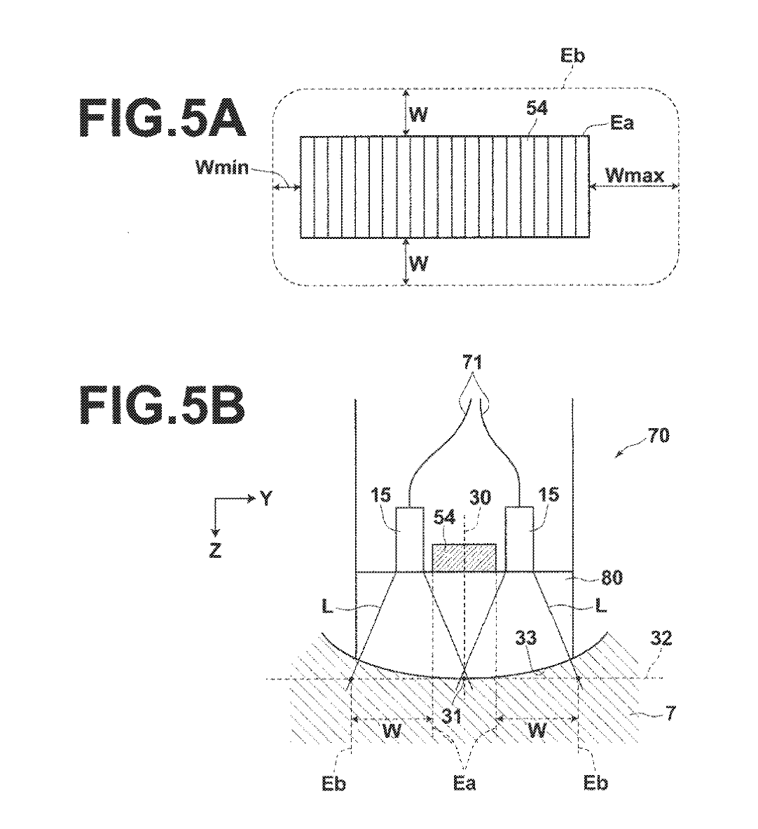

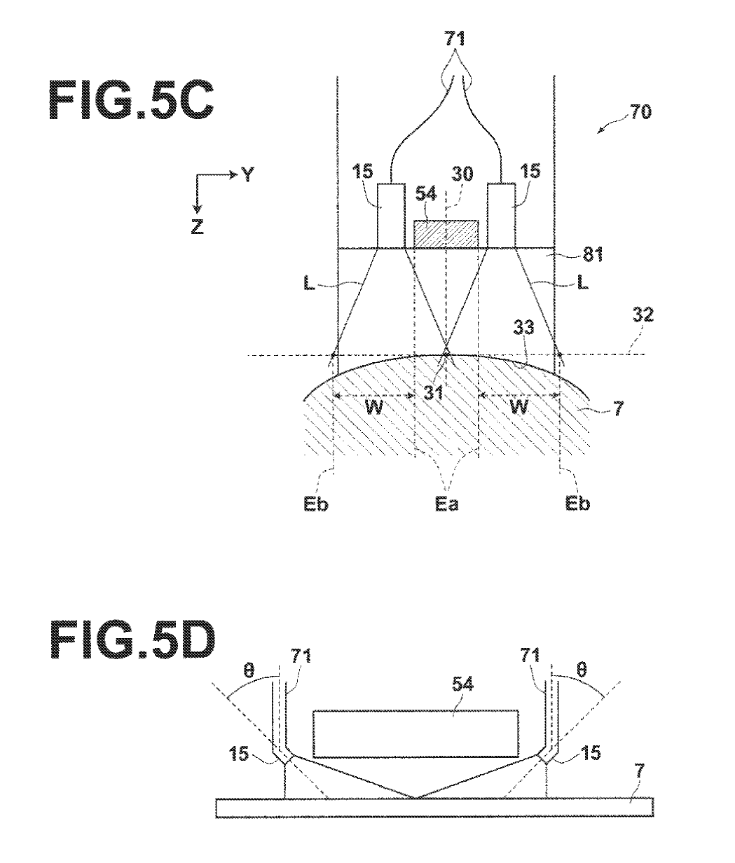

Next, the ultrasound probe 70, in which the light emitting section 15 and the electroacoustic converting section 3 are integrated, will be described with reference to FIG. 3 through FIG. 5C. FIG. 3 is a schematic diagram that illustrates the configuration of the ultrasound probe 70. FIG. 4 illustrates the ultrasound probe 70 and living tissue when generating a photoacoustic image. FIG. 5A through FIG. 5C are schematic diagrams for explaining the positional relationship between the outer peripheral edge of a corresponding region related to one dimensionally arranged converting elements and the outer peripheral edge of an emission range that includes the corresponding region.

The ultrasound probe 70 has the plurality of converting elements 54. The converting elements 54 are arranged one dimensionally along a predetermined direction, for example. In FIG. 3, the direction in which the converting elements 54 are arranged (array direction) is illustrated as an X axis, the direction perpendicular to the array direction and parallel to the detecting surfaces of the converting elements 54 (elevation direction) is illustrated as a Y axis, and the direction perpendicular to the array direction and the elevation direction is illustrated as a Z axis. The optical fibers 71 guide light from the light source section 11 (FIG. 1) to the light emitting section 15 provided within the ultrasound probe 70. As illustrated in FIG. 3, the light emitting section 15 is arranged along the periphery of the one dimensionally arrayed converting elements 54, for example. As illustrated in FIG. 3 and FIG. 5A, the light emitting section 15 is configured such that a pulsed laser beam can be emitted onto an emission range on the subject 7 that includes the entirety of a corresponding region of the subject 7 that corresponds to the electroacoustic converting section 3, the minimum distance Wmin between the outer peripheral edge Ea of the corresponding region and the outer peripheral edge Eb of the emission range being 5 mm or greater. The expression “minimum distance Wmin between the outer edge Ea of the corresponding region and the outer peripheral edge Eb of the emission range” refers to the minimum width from among widths W of an annular region between the outer peripheral edge of the emission range and the outer peripheral edge of the corresponding region. Here, the widths W of the annular region may be determined at arbitrary positions along the outer peripheral edge Ea of the corresponding region or along the outer peripheral edge Eb of the emission range. A width W at a position along the outer peripheral edge Ea of the corresponding region (or the outer peripheral edge Eb of the emission range) is the shortest distance from the position to the outer peripheral edge Eb of the emission range (or the outer peripheral edge Ea of the corresponding region).

In greater detail, in the present specification, the corresponding region on the subject and the emission range are defined on a plane (reference plane) that passes through an intersection between an axis that passes through the center of the electroacoustic converting section and is parallel to the Z axis and an abutment surface where the ultrasound probe abuts the subject, and is perpendicular to the Z axis. The center of the electroacoustic converting section is the center of the shape of the electroacoustic converting section when viewed from a viewpoint along the Z axis. Generally, the electroacoustic converting section is constituted by one dimensionally or two dimensionally arrayed converting elements, and is of a rectangular shape as a whole. Therefore, in such a case, the center of the electroacoustic converting section refers to the center of the rectangular shape. In addition, the abutment surface of the ultrasound probe is the surface of the ultrasound probe that contacts the subject when the ultrasound probe is abutted against the subject. For example, in the case that the detecting surface of the electroacoustic converting section directly contacts the subject, the contact surface between the detecting surface and the subject is the abutment surface. In the case that acoustic elements such as an acoustic rectifying layer and an acoustic lens provided on the ultrasound probe directly contact the subject, the contact surface between the acoustic element and the subject is the abutment surface.

In the case that the abutment surface is curved, the reference plane 32 is defined as illustrated in FIG. 5B and FIG. 5C. FIG. 5B illustrates a case in which an acoustic element 80 having a convex surface is provided at the tip of an ultrasound probe. In the case illustrated in FIG. 5B, the contact surface between the convex surface of the acoustic element 80 and the surface of the subject 7 is the abutment surface 33. Meanwhile, FIG. 5C illustrates a case in which an acoustic element 81 having a concave surface is provided at the tip of an ultrasound probe. In the case illustrated in FIG. 5C, the contact surface between the convex surface of the acoustic element 81 and the surface of the subject 7 is the abutment surface 33. In FIG. 5B and FIG. 5C, the plane that passes through an intersection 31 between an axis 30 that passes through the center of the electroacoustic converting section 30 and are parallel to the Z axis and the abutment surface 33, and is perpendicular to the Z axis, is the reference plane 32. The corresponding region and the emission range are defined on the reference plane 32. That is, the corresponding region is a projection region of the electroacoustic converting section onto the reference plane 32 along the Z axis, and the distance between the outer edge Ea of the corresponding region and the outer edge Eb of the emission range is the width W of the annular region.

The light emitting section 15 may be configured to emit the pulsed laser beam output by the light source section 11 onto a region that includes a selected partial region at least. For example, the light emitting section 15 may be provided corresponding to each of a region A, a region B, and a region C, as illustrated in FIG. 4. In this case, the light emitting section 15 corresponding to the region A emits the pulsed laser beam at least onto the region A when the region A is selected. The light emitting section 15 corresponding to the region B emits the pulsed laser beam at least onto the region B when the region B is selected. The light emitting section 15 corresponding to the region C emits the pulsed laser beam at least onto the region C when the region C is selected. At this time, the light emitting section 15 provided corresponding to each partial region is set such that the minimum distance Wmin between the outer edge Ea of the corresponding region on the subject 7 corresponding to the converting elements 54 within each partial region and the outer edge Eb of the emission range is 5 mm or greater. That is, the light emitting section 15 corresponding to the region A is set such that the minimum distance Wmin between the outer edge Ea of the corresponding region on the subject 7 corresponding to the converting elements 54 which are driven when the pulsed laser beam is emitted onto the region A and the outer edge Eb of the emission range is 5 mm or greater.

The ultrasound probe 70 has converting elements 54 corresponding to 192 channels, for example. Consider a case in which the width corresponding to the converting elements 54 is divided into three partial regions (regions A through C) related to photoacoustic image generation, and the width of each partial region corresponds to converting elements 54 for 64 channels, for example. In this case, if the width of living tissue corresponding to 192 channels is 57.6 mm, the width of each partial region will be 19.2 mm. That is, the photoacoustic imaging apparatus 10 repeats light emission and data collection to and from 19.2 mm wide partial regions divided as illustrated in FIG. 4 three times, to obtain data for all 192 channels.

Note that in the case that the light emitting section 15 is provided about the periphery of the electroacoustic converting section 3 (the arrayed converting elements 54), it is preferable for the emission axes of the light beams emitted by the light emitting section 15 to be inclined at an angle within a range from 5 through 45 degrees toward the corresponding region with respect to a direction perpendicular to the corresponding region, as illustrated in FIG. 5D. This configuration enables shadows of the electroacoustic converting section 3 to be reduced with respect to the emission range of the light beams.

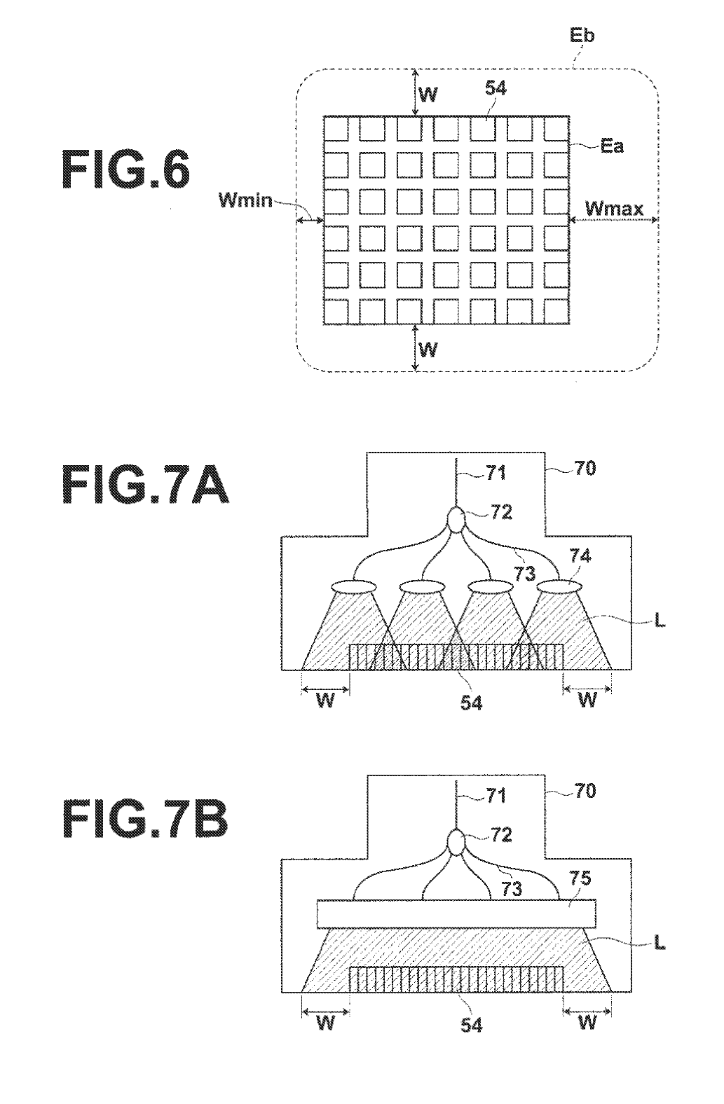

The present invention is not limited to using the electroacoustic converting section 3 in which the converting elements 54 are one dimensionally arrayed. That is, the present invention may also be applied to an electroacoustic converting section 3 in which the converting elements are arranged two dimensionally. In the case that optical fibers are inserted and arranged as illustrated in FIG. 6, the converting elements 54 may be arranged with predetermined intervals therebetween. In such a case, the outer peripheral edge Ea of the corresponding region is a collection of positions along the outer periphery of the converting elements 54 which are arranged as illustrated in FIG. 6 as a whole.

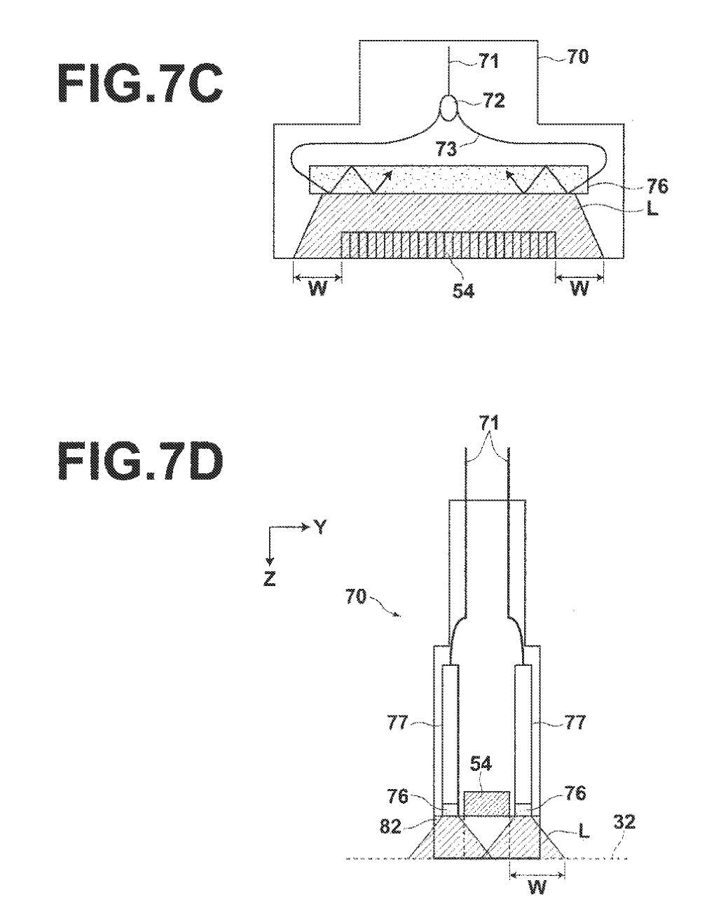

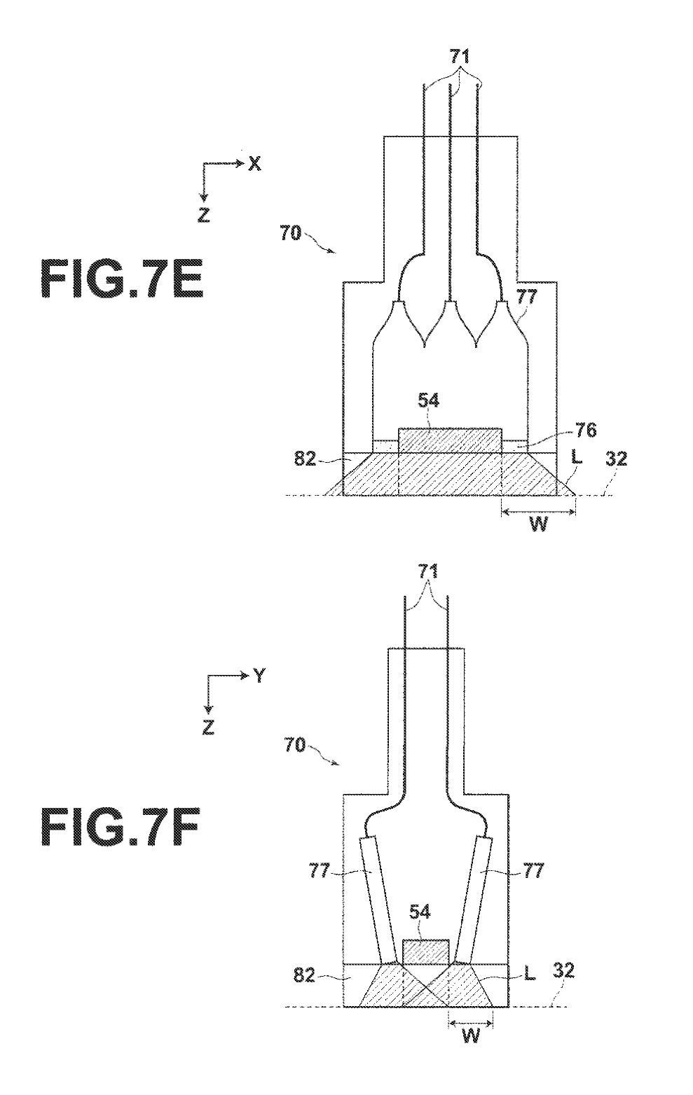

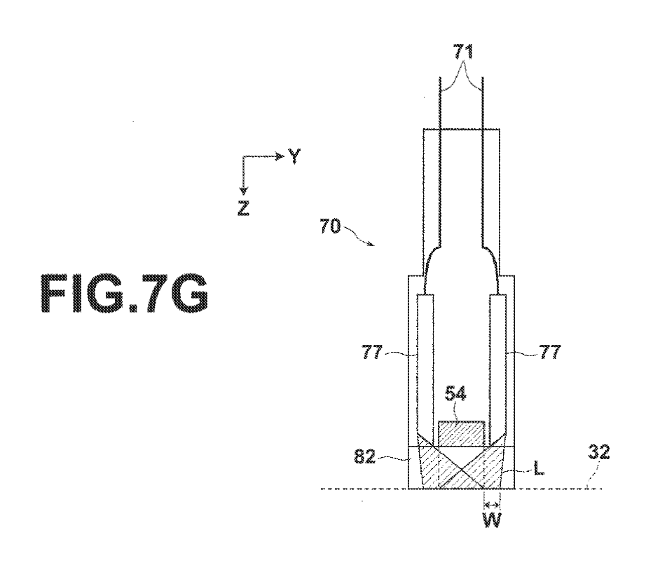

In the case that the light emitting section 15 of the ultrasound probe 70 is configured to enable the entirety of the converting elements 54 to be irradiated from above, the configurations illustrated in FIG. 7A through FIG. 7G are preferable, from the viewpoint of obtaining a uniform light intensity distribution.

Here, FIG. 7A is a schematic diagram that illustrates the structure of a light transmitting section 1 equipped with a combination of an optical fiber 71 as a waveguide section 14, a plurality of optical fibers 73 that branch a light beam from the optical fiber 71 via an optical fiber coupler 72, and a plurality of circular lenses 74. In this structure, the plurality of circular lenses 74 are arrayed along the longitudinal direction of the electroacoustic converting section 3. All of the light beams L output from each of the circular lenses realize an emission range that includes the corresponding region corresponding to the electroacoustic converting section 3. In FIG. 7A, the plurality of circular lenses 74 constitute the light emitting section 15.

FIG. 7B is a schematic diagram that illustrates the structure of a light transmitting section 1 equipped with a combination of an optical fiber 71 as a waveguide section 14, a plurality of optical fibers 73 that branch a light beam from the optical fiber 71 via an optical fiber coupler 72, and a single rectangular lens 75. In this structure, the rectangular lens 75 having a length greater than or equal to the length of the electroacoustic converting section 3 is provided along the longitudinal direction of the electroacoustic converting section 3. The light beam L output from the rectangular lens 75 realizes an emission range that includes the corresponding region corresponding to the electroacoustic converting section 3. A cylindrical lens is an example of a rectangular lens. In FIG. 7B, the rectangular lens 75 constitutes the light emitting section 15.

FIG. 7C is a schematic diagram that illustrates the structure of a light transmitting section 1 equipped with a combination of an optical fiber 71 as a waveguide section 14, two optical fibers 73 that branch a light beam from the optical fiber 71 via an optical fiber coupler 72, and a light diffusing section 76. In this structure, the light diffusing section 76 having a length greater than or equal to the length of the electroacoustic converting section 3 is provided along the longitudinal direction of the electroacoustic converting section 3. The two optical fibers 734 are connected to the two ends of the light diffusing section 76. Light that enters the light diffusing section 76 from the two ends thereof propagate through the light diffusing section 76 and is diffused by diffusing particles within the light diffusing section 76. The light beam L output from the light diffusing section 76 realizes an emission range that includes the corresponding region corresponding to the electroacoustic converting section 3. In FIG. 7C, the light diffusing section 76 constitutes the light emitting section 15.

FIG. 7D is a schematic diagram that illustrates the structure of a light transmitting section 1 equipped with a combination of optical fibers 71 as a waveguide section 14 and light guiding plates 77. FIG. 7E is a schematic diagram that illustrates the ultrasound probe 70 of FIG. 7D from the elevation direction. In this structure, the two light guiding plates 77 having widths greater than or equal to the length of the electroacoustic converting section 3 are provided at the sides of the electroacoustic converting section 3. The two light guiding plates 77 are provided such that the electroacoustic converting section 3 constituted by the plurality of converting elements 54 is sandwiched therebetween and such that they face each other in a direction perpendicular to the direction in which the plurality of converting elements 54 are arrayed. The optical fibers 71 and the light guiding plates 77 are optically coupled. The portions of the light guiding plates 77 at the sides which are coupled to the optical fibers 71 are formed as tapered shapes as illustrated in FIG. 7E, for example. It is preferable for the portions of the light guiding plates 77 which are coupled to the optical fibers 71 to be formed by a glass material, in order to avoid damage caused by light energy. Meanwhile, the other portions of the light guiding plates 77 are formed by a resin material, such as acryl. Pulsed laser beams L which are guided by the optical fibers 71 enter the light guiding plates 77, then are emitted onto the subject 7 from the opposite ends of the light guiding plates 77. Note that in FIG. 7D and FIG. 7E, light diffusing sections 76 are provided at the ends of the light guiding plates 77 through which the pulsed laser beams L are output, and an acoustic element 82 is provided at the tip of the ultrasound probe 70. In FIG. 7D and FIG. 7E, the abutment surface of the ultrasound probe 70 is the reference plane 32. However, the light diffusing sections 76 and the acoustic element 82 are not necessary. In FIG. 7D and FIG. 7E, the pulsed laser beams L which are diffused within the light diffusing sections 76 realize an emission range that includes the corresponding region corresponding to the electroacoustic converting section 3. In FIG. 7D and FIG. 7E, the light guiding plates 77 and the light diffusing sections 76 constitute the light emitting section 15.

FIG. 7F is a schematic diagram that illustrates another structure of a light transmitting section 1 equipped with a combination of optical fibers 71 as a waveguide section 14 and light guiding plates 77. The ultrasound probe 70 of FIG. 7F differs from the ultrasound probe of FIG. 7D and FIG. 7E mainly in that the light guiding plates 77 are inclined with respect to the Z axis such that the pulsed laser beams L output from the light guiding plates 77 are more oriented toward the corresponding region, and that the light diffusing section 76 are not provided. By adopting this configuration, shadows of the electroacoustic converting section 3 can be reduced with respect to the emission range of the light beams.

FIG. 7G is a schematic diagram that illustrates still another structure of a light transmitting section 1 equipped with a combination of optical fibers 71 as a waveguide section 14 and light guiding plates 77. The ultrasound probe 70 of FIG. 7G differs from the ultrasound probe of FIG. 7D and FIG. 7E mainly in that the ends of the light guiding plates 77 through which the pulsed laser beams L are output are cut to have a slope (within a range from 20 through 30 degrees, for example). In the case that the light guiding plates 77 of FIG. 7G are utilized, the pulsed laser beams L will be output toward the corresponding region due to the slope, even if the light guiding plates 77 themselves are not inclined. By adopting this configuration, shadows of the electroacoustic converting section 3 can be reduced with respect to the emission range of the light beams, and the ultrasound probe 70 can be miniaturized.

Note that in cases that the light emitting section 15 is of a configuration as illustrated in one of FIGS. 7A through 7G as well, the light emitting section 15 is set such that the minimum width Wmin from among the widths W of the annular region between the outer peripheral edge of the emission range and the outer peripheral edge of the corresponding region is 5 mm or greater.

Hereinafter, the operative effects of the present invention will be described.

In this embodiment, the light emitting section 15 is configured such that a pulsed laser beam can be emitted onto an emission range on the subject 7 that includes the entirety of the corresponding region of the subject 7 that corresponds to the electroacoustic converting section 3, the minimum distance Wmin between the outer peripheral edge Ea of the corresponding region and the outer peripheral edge Eb of the emission range being 5 mm or greater, as illustrated in FIG. 5A. By adopting this setting, photoacoustic waves having greater intensities can be positively generated from the same tissue system if the light energy density is the same.

This is because in the case that the light energy density is equal, the intensity of photoacoustic waves which are generated from a tissue system increases accompanying an increase in the emission range of light up to a width W of approximately 20 mm between the outer peripheral edge of the emission range and the outer peripheral edge of the corresponding region, as will be proven by an embodiment to be described later. Note that if the aforementioned width W exceeds 20 mm, the intensity of photoacoustic waves approaches a saturated state. Therefore, it is preferable for the maximum width Wmax to be 20 mm or less, from the viewpoint of efficiently utilizing light energy. In addition, if the aforementioned width W is less than 5 mm, the intensity of photoacoustic waves generated in a tissue system will be 1/10 or less the intensity of photoacoustic waves generated in the same tissue system in a saturated state. Accordingly, the minimum width Wmin is at least 5 mm in the present invention, form the viewpoint of detecting photoacoustic waves at a high S/N ratio.

As described above, the photoacoustic inspection probe and the photoacoustic inspection apparatus of the present invention are to be employed in photoacoustic inspection that utilizes the photoacoustic effect, and are equipped with the light emitting section that emits the light beam onto the subject and the electroacoustic converting section that converts the photoacoustic waves into electrical signals. Particularly, the light emitting section is configured to emit light onto an emission range on the subject that includes the entirety of a corresponding region of the subject that corresponds to the electroacoustic converting section, the minimum distance between the outer peripheral edge of the corresponding region and the outer peripheral edge of the emission range being 5 mm or greater. Therefore, photoacoustic waves having greater intensities can be generated from tissue systems in cases that the light energy density is the same. As a result, photoacoustic waves can be detected with higher S/N ratios in photoacoustic inspection that utilizes the photoacoustic effect.

### Embodiments

An embodiment of the photoacoustic inspection apparatus of the present invention will be described below.

### <Method for Measuring Signal Intensity>

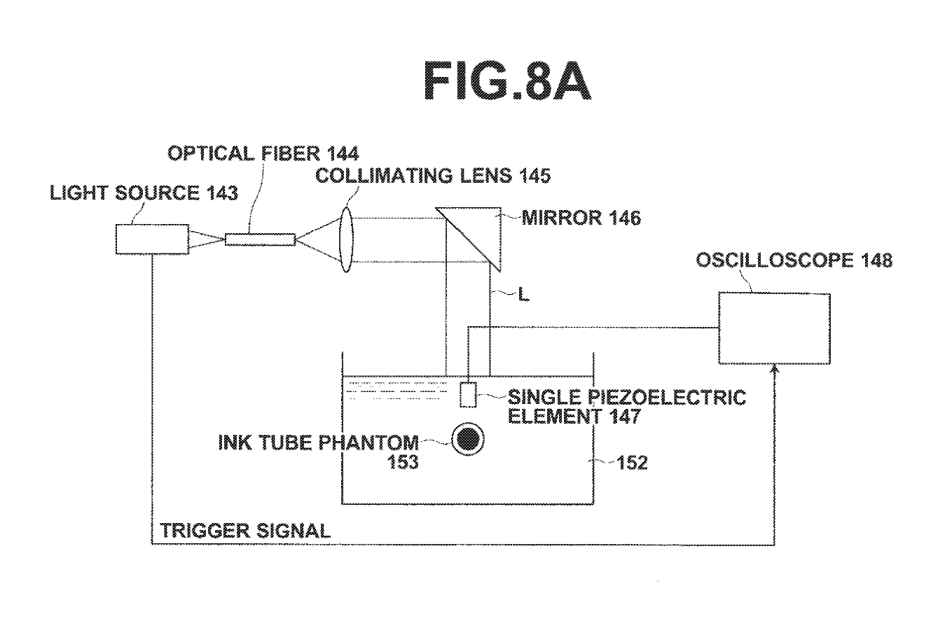

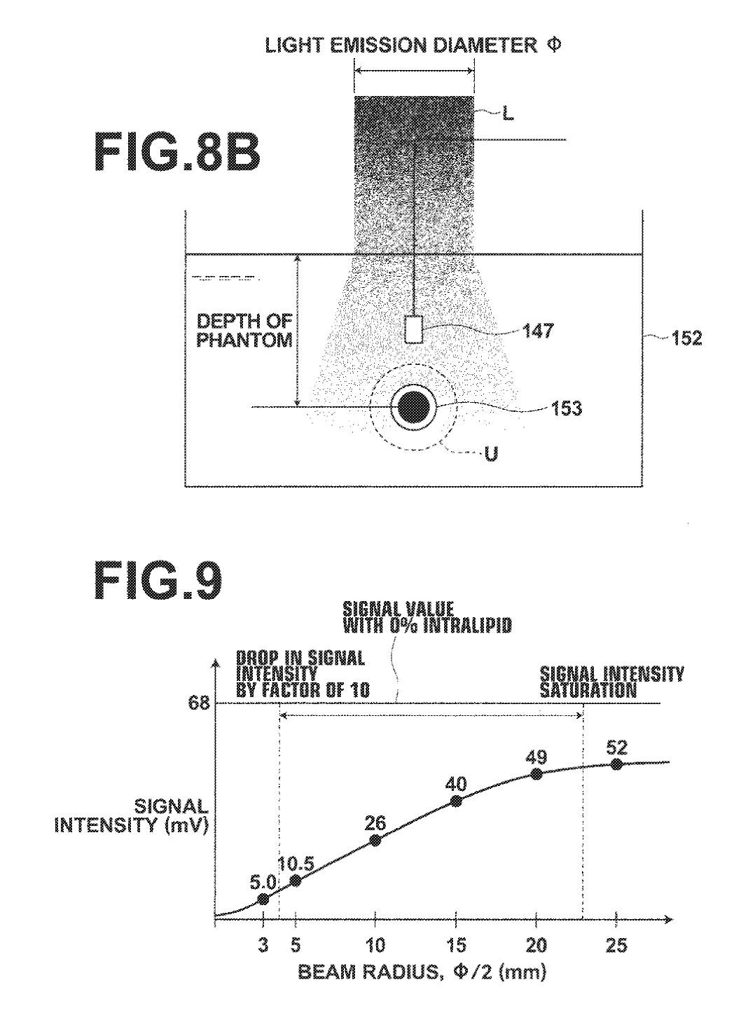

FIG. 8A and FIG. 8B are schematic diagrams that illustrate the structure of an apparatus according to the embodiment of the present invention. Measurement was performed with a urethane tube phantom 153 having an inner diameter of 2.5 mm, placed in degassed water 152 having a light scattering substance (Intralipid 0.03%: corresponding to the scattering intensity of living tissue) therein, as a target. The urethane tube phantom was placed at a position at which the center thereof was 15 mm from the surface of the water. Ink, having the same coefficient of light absorption as blood within living tissue, was sealed within the urethane tube phantom 153.

A laser beam having a wavelength of 532 nm was output from a YAG excited OPO laser, guided by an optical fiber 144, collimated by a collimating lens 145, reflected by a mirror 146 as a collimated laser beam such that the emission axis was perpendicular to the surface of the water, then emitted onto the urethane tube phantom 153 as a laser beam L. A PVDF single piezoelectric element 147 provided in the water close to the surface and within the emission range of the laser beam L detected photoacoustic waves U from the urethane tube phantom 153. The signal waveforms of the photoacoustic waves U were observed with an oscilloscope 148, and voltage intensities were designated as signal intensities of the photoacoustic waves U. In this case, the corresponding region of the present invention was treated as a region on the surface of the water. The light energy density of the laser beam L was maintained constant at 0.5 J/cm², while the emission range was varied by changing the beam diameter Φ (mm) at the surface of the water. Changes in the intensity of the photoacoustic waves U were evaluated at each of the beam diameters Φ.

### <Results>

FIG. 9 is a graph that illustrates the measurement results obtained by the embodiment. The vertical axis represents signal intensities (mV) of the photoacoustic waves U, and the horizontal axis represents radii Φ/2 (mm) of the laser beam. From this graph, it can be understood that the signal intensity of the photoacoustic waves U became substantially saturated when the beam radius exceeds 20 mm, that is, when the distance from the position of the outer peripheral edge of the emission range of light to the single piezoelectric element 147 exceeds 20 mm. Meanwhile, the signal intensity of the photoacoustic waves U decreased by a factor of 10 when the beam radius is less than 5 mm, that is, when the distance from the position of the outer peripheral edge of the emission range of light to the single piezoelectric element 147 is less than 5 mm. Accordingly, the signals of the photoacoustic waves U became buried in noise and detection became difficult when the beam radius was less than 5 mm.

As a result, it can be said that the minimum distance between the outer peripheral edge of the corresponding region and the outer peripheral edge of the emission range needs to be set to be 5 mm or greater in order to positively detect photoacoustic waves at high S/N ratios in photoacoustic inspection that utilizes the photoacoustic effect. Further, it can be said that the advantageous effects of the present invention can be sufficiently obtained even if the maximum distance between the outer peripheral edges is 20 mm or less.

What is claimed is:

1. A photoacoustic inspection probe, comprising:
a light emitting section that emits a light beam onto a subject; and
an electroacoustic converting section that converts photoacoustic waves, which are generated within the body of the subject by the light beam being emitted thereon, into electrical signals;
the light emitting section being configured to emit light onto an emission range on the subject that includes the entirety of a corresponding region of the subject that corresponds to the electroacoustic converting section, the minimum distance between the outer peripheral edge of the corresponding region and the outer peripheral edge of the emission range being 5 mm or greater and the maximum distance between the outer peripheral edge of the corresponding region and the outer peripheral edge of the emission range being 20 mm or less.

2. A photoacoustic inspection probe as defined in claim 1, wherein:
the light emitting section comprises a plurality of light guiding sections that branch the light beam, arranged at constant intervals.

3. A photoacoustic inspection probe as defined in claim 1, wherein:
the light emitting section comprises a plurality of combinations of a light guiding section that branches the light beam and a circular lens.

4. A photoacoustic inspection probe as defined in claim 1, wherein:
the light emitting section comprises a combination of a plurality light guiding sections provided at constant intervals that branch the light beam, and a rectangular lens.

5. A photoacoustic inspection probe as defined in claim 1, wherein:
the light emitting section comprises a light diffusing section for causing light having a substantially uniform intensity to be emitted within the emission range.

6. A photoacoustic inspection probe as defined in claim 1, wherein:
the emission axis of the light beam emitted by the light emitting section is inclined at an angle within a range from 5 through 45 degrees toward the corresponding region with respect to a direction perpendicular to the corresponding region.

7. A photoacoustic inspection probe as defined in claim 1, wherein:
the light emitting section is a pair of light guiding plates which are provided to face each other with the electroacoustic converting section interposed therebetween.

8. A photoacoustic inspection probe as defined in claim 7, wherein:
the light guiding plates have light diffusing sections at the end portions thereof through which light is output.

9. A photoacoustic inspection probe as defined in claim 7, wherein:
the end portions of the light guiding plates through which the light beam is output are cut so as to have a slope.

10. A photoacoustic inspection probe as defined in claim 9, wherein:
the slope is angled within a range from 20 through 30 degrees.

11. A photoacoustic inspection probe as defined in claim 9, wherein:
the light guiding plates are parallel to an axis perpendicular to a detecting surface of the electroacoustic converting section.

12. A photoacoustic inspection probe as defined in claim 9, wherein:
an acoustic element is provided on a detecting surface of the electroacoustic converting section.

13. A photoacoustic inspection apparatus, comprising:
a light source that generates a light beam to be emitted onto a subject;
a light emitting section that emits the light beam onto the subject;
an electroacoustic converting section that converts photoacoustic waves generated within the body of the subject by the light beam being emitted thereon and converts the photoacoustic waves into electrical signals; and
an image generating section that generates photoacoustic images based on the electrical signals;
the light emitting section being configured to emit light onto an emission range on the subject that includes the entirety of a corresponding region of the subject that corresponds to the electroacoustic converting section, the minimum distance between the outer peripheral edge of the corresponding region and the outer peripheral edge of the emission range being 5 mm or greater and the maximum distance between the outer peripheral edge of the corresponding region and the outer peripheral edge of the emission range being 20 mm or less.

14. A photoacoustic inspection apparatus as defined in claim 13, wherein:
the light emitting section is a pair of light guiding plates which are provided to face each other with the electroacoustic converting section interposed therebetween.

15. A photoacoustic inspection apparatus as defined in claim 14, wherein:
the end portions of the light guiding plates through which the light beam is output are cut so as to have a slope.

16. A photoacoustic inspection apparatus as defined in claim 15, wherein:
the slope is angled within a range from 20 through 30 degrees.

17. A photoacoustic inspection apparatus as defined in claim 15, wherein:
the light guiding plates are parallel to an axis perpendicular to a detecting surface of the electroacoustic converting section.

18. A photoacoustic inspection apparatus as defined in claim 15, wherein:
an acoustic element is provided on a detecting surface of the electroacoustic converting section.
